(19) United States
(12) Patent Application Publication
(10) Pub. No.: US 2025/0248824 A1
(43) Pub. Date: Aug. 7, 2025

(54) INTERVERTEBRAL IMPLANT HAVING VARIABLE HEIGHT SERRATIONS

(71) Applicant: Additive Implants, Inc., Phoenix, AZ (US)

(72) Inventors: John R. Ehteshami, Paradise Valley, AZ (US); Matt Zoghi, Phoenix, AZ (US)

(73) Assignee: Additive Implants, Inc., Phoenix, AZ (US)

(21) Appl. No.: 19/043,052

(22) Filed: Jan. 31, 2025

Related U.S. Application Data
(60) Provisional application No. 63/548,784, filed on Feb. 1, 2024.

(51) Int. Cl. A61F 2/44; A61F 2/30

(52) U.S. Cl. CPC A61F 2/4455; A61F 2002/30322; A61F 2002/30326; A61F 2002/30784; A61F 2002/30904; A61F 2002/3093

(57) ABSTRACT

The present invention relates to an intervertebral implant having serrations with varying heights above the upper and/or lower surfaces. The intervertebral implant may include serrations with different angles of inclination arising from the upper and lower surfaces

### CROSS REFERENCE TO RELATED APPLICATIONS

The application claims priority to, and the benefit of, U.S. Provisional Patent Application Ser. No. 63/548,784 filed on Feb. 1, 2024 entitled “INTER VERTEBRAL IMPLANT HAVING VARIABLE HEIGHT SERRATIONS”, the disclosure of which is hereby incorporated by reference in their entirety.

### FIELD OF THE INVENTION

The present disclosure generally relates to fixation devices and systems for positioning and immobilizing at least two adjacent vertebrae and methods related to the same. In particular, the present disclosure relates to interbody fusion devices with an integrated solid support structure and porous ingrowth structure.

### BACKGROUND OF THE INVENTION

The human spinal column has more than twenty discrete bones sequentially coupled to one another by a tri-joint complex that consists of an anterior disc and two posterior facet joints, the anterior discs of adjacent bones being cushioned by cartilage spacers referred to as intervertebral discs. The more than twenty bones are anatomically categorized in one of four classifications: cervical, thoracic, lumbar, or sacral. The cervical portion of the spine extends from the base of the skull and includes the first seven vertebrae. The intermediate twelve vertebrae make up the thoracic portion of the spine. The lower portion of the spine comprises five lumbar vertebrae. The base of the spine is the sacral bones (including the coccyx). The component bones of the cervical spine are generally smaller than those of the thoracic spine, which are in turn smaller than those of the lumbar region. The sacral region connects laterally to the pelvis. While the sacral region is an integral part of the spine, for the purposes of fusion surgeries and for this disclosure, the word spine shall refer only to the cervical, thoracic, and lumbar regions.

The spinal column is highly complex in that it includes these more than twenty bones coupled to one another, housing and protecting critical elements of the nervous system having innumerable peripheral nerves and circulatory bodies in dose proximity. In spite of these complications, the spine is a highly flexible structure, capable of a high degree of curvature and twist in nearly every direction.

The intervertebral disc disposed between the vertebrae in the human spine has a peripheral fibrous shroud (the annulus) that surrounds a spheroid of flexibly deformable material (the nucleus). The nucleus comprises a hydrophilic, elastomeric cartilaginous substance that cushions and supports the separation between the bones. The nucleus also permits articulation of adjacent vertebral bones relative to one another to the extent such articulation is allowed by the other soft tissue and bony structures surrounding the disc. The additional bony structures that define pathways of motion in various modes include the posterior joints (the facets) and the lateral intervertebral joints (the unco-vertebral joints). Soft tissue components, such as ligaments and tendons, constrain the overall segmental motion as well.

Traumatic, genetic, and long-term wearing phenomena contribute to the degeneration of the nucleus in the human spine. This degeneration of this critical disc material, from the hydrated, elastomeric material that supports the separation and flexibility of the vertebral bones, to a flattened and inflexible state, has profound effects on the mobility (instability and limited ranges of appropriate motion) of the segment, and can cause significant pain to the individual suffering from the condition. Although the specific causes of pain in patients suffering from degenerative disc disease of the cervical spine have not been definitively established, it has been recognized that pain may be the result of neurological implications (nerve fibers being compressed) and/or the subsequent degeneration of the surrounding tissues (the arthritic degeneration of the facet joints) as a result of their being overloaded.

Traditionally, the treatment of choice for physicians caring for patients who suffer from significant degeneration of the cervical intervertebral disc is to remove some, or all, of the damaged disc. In instances in which a sufficient portion of the intervertebral disc material is removed, or in which much of the necessary spacing between the vertebrae has been lost (significant subsidence), restoration of the intervertebral separation is required.

Unfortunately, until the advent of spine arthroplasty devices, the only methods known to surgeons to maintain the necessary disc height necessitated the immobilization of the segment. Immobilization is generally achieved by attaching metal plates to the anterior or posterior elements of the cervical spine, and the insertion of some osteoconductive material (autograft, allograft, or other porous material) between the adjacent vertebrae of the segment. This immobilization and insertion of osteoconductive material has been utilized in pursuit of a fusion of the bones, which is a procedure carried out on tens of thousands of pain suffering patients per year.

This sacrifice of mobility at the immobilized, or fused, segment, however, is not without consequences. It was traditionally held that the patient's surrounding joint segments would accommodate any additional articulation demanded of them during normal motion by virtue of the fused segment's immobility. While this is true over the short-term (provided only one, or at most two, segments have been fused), the effects of this increased range of articulation demanded of these adjacent segments has become a concern. Specifically, an increase in the frequency of returning patients who suffer from degeneration at adjacent levels has been reported.

Whether this increase in adjacent level deterioration is truly associated with rigid fusion, or if it is simply a matter of the individual patient's predisposition to degeneration is unknown. Either way, however, it is clear that a progressive fusion of a long sequence of vertebrae is undesirable from the perspective of the patient's quality of life as well as from the perspective of pushing a patient to undergo multiple operative procedures.

In many cases, to alleviate back pain from degenerated herniated discs, the disc is removed along with all or part of at least one neighboring vertebra and is replaced by an implant that promotes fusion of the remaining bony anatomy. However, the success or failure of spinal fusion may depend upon several factors. For instance, the spacer or implant or cage used to fill the space left by the removed disc and bony anatomy must be sufficiently strong to support the spine under a wide range of loading conditions. The spacer should also be configured so that it will likely remain in place once it has been positioned in the spine by the surgeon. Additionally, the material used for the spacer should be biocompatible material and should have a configuration that promotes bony ingrowth.

Proper fixation and stability is an important factor in the development of stand-alone anterior lumbar interbody fusion (ALIF) devices. Improving the design of ALIF device surface architectures could lead to significant advances in the development of a device capable of stand-alone use without the addition of a 360° surgery for additional posterior fixation.

Spondylolisthesis is a debilitating, degenerative condition of the lumbar spine in which the superior lumbar vertebra translates anteriorly with respect to the inferior vertebra. While several fusion devices are currently available, severe cases often require 360° surgery, i.e., a combined anterior/posterior approach, to restore anatomic alignment and alleviate pain. This procedure is both invasive and expensive. The present study is part of a series of ongoing in vitro evaluations of surface architecture for a novel standalone 3D-printed interbody device. In this study, we examined the effects of surface architecture on interface micromotion under simulated physiological loading.

Every year there are approximately 266 million new cases of lumbar degenerative spinal disease worldwide. The direct and indirect costs to treat these patients in the US alone have been estimated to exceed $600 billion. The volume of lumbar fusion surgeries attributed to degenerative diseases has skyrocketed over the last two decades, with estimates of the increase ranging from 62%-118%. A typical spine fusion procedure involves the surgical use of sharp dissection instruments, such as curettes and rasps, to remove the diseased disc and scrape off the vertebral endplate cartilage. Once the vertebral endplates have been debrided of cartilage and a bleeding bony surfaces are achieved on the adjacent vertebral end plates, an interbody fusion device (IBFD) or cage packed with allograft or autograft bone, is inserted into the space previously occupied by the diseased disc. This IBFD acts as scaffolding to facilitate bony fusion between the two adjacent vertebral bodies. Other than host factors, the success of a spinal fusion is predominantly driven by two factors: biological healing ability of the bone graft, as well as the IBFD and mechanical stability between vertebral bodies. For osteogenesis to occur, osteoblasts must be stimulated to produce bone tissue. Additionally, the bone-implant-bone construct must have sufficient stability to promote fusion. Maximum construct stability is essential in intra-vertebral fusion as compared to long bone healing, such as the femur. Therefore, micromotion among all components of the structure (vertebrae, IBFD, and additional stabilizing hardware) must be sufficiently minimized, ideally below 50 microns, to facilitate osseointegration between the implant and the host bone.

Currently, there are several stand-alone anterior lumbar interbody fusion devices for the surgical treatment of spondylolisthesis on the market, intended to minimize surgical cost and invasiveness and maximize lumbar stability. However, no studies to date have investigated the importance of surface microarchitecture on device fixation.

In the United States, approximately 60% of lumbar fusion procedures, or roughly 120,000 surgeries, are performed annually for the surgical correction of spondylolisthesis. Spondylolisthesis is a slippage of adjacent vertebra in the sagittal plane, due to shear forces. This slippage stems from disc degeneration, which narrows the disc space and predisposes the spine to instability in the sagittal plane. Spondylolisthesis is categorized in five grades, with Level I being the least severe and subsequent grades being more severe. An increase in grade correlates to an increase in the complexity of the operation to address the pathology, as well as an increase in the forces experienced by the stabilizing devices. Spondylolisthesis commonly affects individuals 50 years and older, with an increased prevalence in overweight and obese patients and in women, largely associated with complications from previous pregnancies. This condition places the individual at risk for nerve root compression and can cause lower back pain and/or sciatica that can obstruct common daily activities. Other disease manifestations include facet hypertrophy, ligament ossifications, and neurological claudicationCurrent surgical strategies include decompression, fusion, or a combination of the two with instrumentation to prevent slippage and improve fusion rate. Limitations of these techniques include complications such as none-union requiring reoperation. In summary, spondylolisthesis is a serious condition that affects the lives of millions of patients worldwide. The excessive costs of doctor visits, physical therapy, medications, surgery, and the lack of consistently effective treatment for this condition, place a heavy socioeconomic burden on the community.

There are two dominant surgical approaches for achieving fusion for the treatment of spondylolisthesis: Anterior Lumbar Interbody Fusion (ALIF) through an anterior retroperitoneal approach, and Transforaminal Lumbar Interbody Fusion (TLIF) through a posterior approach. Both approaches are widely used, as each offers unique advantages, based on varying factors and have different risks and benefits. TLIF is commonly used at L3-L4 and L4-L5 levels, with occasional use at the L5-S1 level; however, the TLIF interbody devices have smaller footprints compared to ALIF implants, and thus provide significantly less mechanical stability as compared to ALIFs. Further, both nerve root anatomy, and endplate morphology differs at L5-S1, which is more ovular, and has a larger lordotic angle. The anatomy at this level contributes to a suboptimal contact area for fixation, complicating this approach compared to higher levels on the lumbar spine, and greater risk of nerve root injury.

The number of ALIF approaches has been increasing significantly in the past several years for several different reasons. ALIF procedures are widely used for treatment of spondylolisthesis at L5-S1, and more severe cases at higher levels, for which TLIF would not provide sufficient mechanical stability. One major advantage of an ALIF approach, is that it avoids the posterior muscles and neural structures which decreases the incidence of their collateral damage during cage insertion. Bypassing these structures allows the surgeon to place a larger footprint cage. The large cage covers a greater surface area of the vertebral body than what can be achieved in TLIF procedures, which allows for greater distribution of load over the spinal endplates. The greater distribution of load leads to ALIF approaches resulting in the lowest occurrence of implant subsidence of all approaches. The capability of a large device also restores disc space height and lumbar lordosis, indirectly decompressing the nerve roots. Satomi et al., reported that ALIF alone achieved reduction of spondylolisthesis from 18.5 to 7.5%. Recent studies have also shown higher rates of fusion in ALIF procedures as well as less total surgical blood loss. Therefore, an optimized ALIF IBFD holds significant promise for safe use in the lower lumbar spine for reduction and stabilization of spondylolisthesis, decreasing the risk of non-union, and increased ability to restore normal sagittal alignment.

However, the majority of ALIF procedures for severe spondylolisthesis require additional posterior fixation (rods and pedicle screws) to stabilize the spine.) This combined anterior-posterior surgery is referred to as a “360° fusion.” A fusion that requires both an anterior and a posterior approach is not only expensive, but comes with all the added risks associated with increased operating times, hospitalization, and blood loss, and post-operative pain. Further, there have been many complications reported with posterior approaches including nerve and paraspinal muscle injury, and infection.

All surgical approaches for spinal fusion, including standalone ALIF, share a risk for sacral insufficiency fractures (SIF). Risk factors such as osteoporosis, osteopenia, or obesity increase the likelihood of postoperative anterior S1 fracture due to combined compressive and shear stresses being concentrated on the interface between the anterior portion of the implant and S1. Incidence of SIF is relatively lower in short segment fusions (<2 segments), but has a 14.5% incidence in fusion over three vertebra segments. Due to the unique anatomy of the L5-S1 vertebra, the only structure resisting a large portion of the shear and compressive stress of common fusion cages is the trabecular bone beneath the anterior portion of the device. This concentration of stress on a small segment of bone is a major design flaw that isn't accounted for in most fusion cage designs and can lead to the need for revision surgeries in a significant number of patients.

The need to avoid the combined anterior and posterior approach has led to the development of “standalone” ALIF devices. These IBFDs have inter-fixated anchors, usually screws that secure the device to the adjacent vertebrae, which aid in stabilization. The screws are intended to minimize motion between the healing surfaces and to prevent cage migration. Due to the increased stability of the integrated screw design, standalone devices for the lumbar and cervical spine are intended to provide sufficient mechanical fixation without the need for supplemental posterior hardware. This is thought to be particularly advantageous, as the need for posterior fixation at this level has been reported to increase incidence of neurovascular injury and reoperation. Rao et al. found an overall clinical success rate of 93% when stand-alone ALIF was used to treat low grade degenerative spondylolisthesis. These results have yet to be reproduced by other groups, as many anterior-only IBFDs lack the ability to generate comparable stability without supplemental posterior fixation, substantially limiting their use as sole fixation devices. Recently, more studies have compared standalone ALIF devices to their counterparts with supplemental screw fixation. Tsantrizos et. al compared segmental stability achieved by various standalone IBFDs in cadaver specimens and found range of motion was decreased by 63% in flexion/extension and 69% in lateral bending. They hypothesized that reported residual motion was due to micromotion at the cage-endplate interface. Additionally, an increase in the neutral zone was observed, which supports concerns about initial segmental instability.

While the relative instability of standalone devices is not inherently poor, the degree of stability required to achieve fusion is unknown, thus, many surgeons are apprehensive about using standalone devices without supplemental posterior fixation with pedicle screws and rods, particularly in cases with higher sacral slopes and/or slippage, where the shear forces are higher. Although the potential to reduce operative time and costs by eliminating the need for additional posterior surgery and hardware makes this technology promising, the clinical success, even in the short term, remains to be demonstrated.

Currently, the majority of IBFDs and cages are fabricated from Poly Ether Ether Ketone (PEEK) which is a semi crystalline thermoplastic, or Ti-6Al-4V (titanium alloy). The primary advantages of PEEK as an implant material are that they are relatively inexpensive to manufacture, radiolucent, and offer a lower modulus of elasticity than conventional metallic devices. In contrast, titanium alloy implants have a higher modulus of elasticity and are radiopaque which can hinder clinical radiographic monitoring of healing progress. On the other hand, one major advantage of titanium is that it is highly biocompatible, with decades of successful clinical use for dental implants and larger orthopaedic joints. Given the mechanical strength requirements needed for ALIF standalone devices to resist the high compressive and shear forces involved, titanium alloy is a suitable material for this application and provides the greatest chance of successful fusion.

Sacral fracture after lumbosacral fusion has been reported in the surgical literature. The authors of the reports presumed osteoporosis to be an important risk factor based on patient demographics (elderly women). Obesity has also been theorized as a risk factor because of the increased load at adjacent segments after multisegmental fusion. Mechanical factors, such as iliac crest graft harvesting, number of segments fused as a measure of the length of the lever arm, and balance of the spine in the sagittal plane, also have been suggested. However, the reports have focused on treatment and outcome rather than prevention of these fracture. The current embodiment proposes specific elements of implant that can better distribute the load on the sacrum or other bone to prevent fracturing.

There is a relationship between implant surface architecture and resistance to shear forces, which can be extremely high and a source of catastrophic failure in the lumbar spine. Sacral fractures following lumbar fusion are a very serious complication that can result from excessive loading on the anterior portion of the sacrum. Accordingly, our innovative approach aims to distribute more of the load to the posterior portion of the vertebral body, through increased integration of the IBFD with the posterior aspect of the vertebral endplate, transferring a greater amount of the shear forces to the posterior portion of the vertebra, as opposed to the fracture-prone anterior portion.

There are multiple clinical studies correlating increases in overall construct stability, (i.e. decreased motion between bone segments) directly leading to improved fusion rates. It stands to reason that improved clinical outcomes could be achieved by enhanced fixation that increases construct and implant-bone interface stability, through enhanced resistance to shear forces.

The current embodiment is a unique non symmetrical surface architecture with posterior facing serrations to prevent the superior vertebral body from slipping anteriorly under shear forces seen in normal daily activities. Further, this design feature helps to secure the superior vertebra in the proper sagittal anatomic position during surgery. Similarly, the caudal (bottom) surface has serrations that point anteriorly and includes a deep posterior hook, to prevent the implant from sliding anteriorly. The posterior anchor (identified by the red arrow) secures the posterior aspect of the implant to the strongest portion of the vertebral body, aiding in resisting shear forces as well as forces seen during flexion (forward bending).

The objective of the present invention is to provide an intervertebral implant that is more secure due to a plurality of serrations along the inferior and superior sides, the serrations having various heights along those planes. The heights are defined as the distance from the tip of the serration (or tooth) from the lowest point on the surface adjacent the tooth.

These and other objectives will become apparent from the following description of the invention.

### SUMMARY OF THE INVENTION

In an embodiment, an intervertebral implant for implantation in an intervertebral space between vertebrae, the implant comprising a body extending from an upper surface to a lower surface, the body having an anterior end, a posterior end, and a pair of spaced apart first and second side walls extending between the anterior end and the posterior end, such that an internal chamber is defined within the anterior and posterior ends and the first and second side walls, wherein the body further includes a porous structure configured for bone ingrowth the upper surface further comprising a plurality of upper serrations positioned such that the serrations are angled and directed toward the posterior end of the body wherein the plurality of upper serrations have different heights above the upper surface, and the lower surface further comprising a plurality of lower serrations positioned such that the serrations are angled and directed toward the anterior end of the body, wherein the plurality of lower serrations have different heights above the lower surface. The intervertebral implant wherein the plurality of lower serrations gradually increase in height along the anterior to posterior direction. The intervertebral implant wherein the plurality of lower serrations gradually increase in height by 50% or greater along the anterior to posterior direction. The intervertebral implant wherein the plurality of lower serrations have a variation of height along the anterior to posterior direction by 50% or greater. The intervertebral implant wherein the plurality of lower serrations gradually increase in height along the anterior to posterior direction, such that a posterior-most serration has a height that is 50% greater than the height of an anterior-most serration. The intervertebral implant wherein the plurality of lower serrations gradually increase in height along the anterior to posterior direction, such that a posterior-most serration has a height that is at least 50% greater than the height of an anterior-most serration. The intervertebral implant wherein the plurality of upper serrations gradually increase in height along the posterior to anterior direction. The intervertebral implant wherein the plurality of lower serrations gradually increase in height by 50% or greater along the posterior to anterior direction. The intervertebral implant wherein the plurality of lower serrations have a variation of height along the posterior to anterior direction by 50% or greater. The intervertebral implant wherein the plurality of lower serrations gradually increase in height along the posterior to anterior direction, such that an anterior-most serration has a height that is 50% greater than the height of a posterior-most serration. The intervertebral implant wherein the plurality of lower serrations gradually increase in height along the posterior to anterior direction, such that an anterior-most serration has a height that is at least 50% greater than the height of a posterior-most serration. An intervertebral implant for implantation in an intervertebral space between vertebrae comprising a body extending from an upper surface to a lower surface, the body having an anterior end, a posterior end, and a pair of spaced apart first and second side walls extending between the anterior end and the posterior end, such that an internal chamber is defined within the anterior and posterior ends and the first and second side walls. The body further includes a porous structure configured for bone ingrowth; the upper surface further comprising a plurality of upper serrations. The upper serrations rise from the upper surface at a 45-degree angle, and wherein each of the plurality of upper serrations have different heights above the upper surface. The lower surface further comprising a plurality of lower serrations, wherein each of the plurality of lower serrations extends from the lower surface at a 60-degree angle and are directed toward the anterior end of the body, and wherein the plurality of lower serrations has different heights above the lower surface. The plurality of lower serrations has a variation of height along the anterior to posterior direction of 50% or greater. The plurality of lower serrations gradually increases in height along the anterior to posterior direction, such that a posterior-most serration has a height that is at least 50% greater than the height of an anterior-most serration. The plurality of upper serrations is comprised of teeth. The plurality of lower serrations is comprised of teeth. An intervertebral implant for implantation in an intervertebral space between vertebrae comprising a body extending from an upper surface to a lower surface, the body having an anterior end, a posterior end, and a pair of spaced apart first and second side walls extending between the anterior end and the posterior end, such that an internal chamber is defined within the anterior and posterior ends and the first and second side walls, wherein the body further includes a porous structure configured for bone ingrowth. The upper surface further comprising a plurality of upper serrations that rise from the upper surface at a 45-degree angle, and wherein each of the plurality of upper serrations have different heights above the upper surface. The lower surface further comprising a plurality of lower serrations wherein each of the plurality of lower serrations extends from the lower surface at a 60-degree angle and are directed toward the anterior end of the body. The plurality of lower serrations has different heights above the lower surface. The plurality of lower serrations has a variation of height along the anterior to posterior direction of 50% or greater and the variation of height increases along the anterior to posterior direction, such that a posterior-most serration has a height that is at least 50% greater than the height of an anterior-most serration. The plurality of upper serrations is comprised of teeth. The plurality of lower serrations is comprised of teeth. The plurality of upper serrations is arranged in a non-linear pattern.

### BRIEF DESCRIPTION OF THE DRAWINGS

Exemplary embodiments of the invention will become more fully apparent from the following description and appended claims, taken in conjunction with the accompanying drawings. Understanding that these drawings depict only exemplary embodiments and are, therefore, not to be considered limiting of the invention's scope, the exemplary embodiments of the invention will be described with additional specificity and detail through use of the accompanying drawings in which:

FIG. 1 shows a representative side view of serrations having different angles.

FIG. 2 shows the relationship between sagittal migration in types of bone and shape of serrations on an implant.

FIG. 3 shows a representative side view of serrations having different angles and heights.

FIG. 4 shows the relationship between cyclical micromotion and shape and height of serrations on an implant.

FIG. 5 shows a representation of micromotion testing.

FIG. 6 shows a midsection view of an implant having serrations of different heights.

FIG. 7 shows the top or cranial plane of an implant from the anterior perspective, having serrations of different heights.

FIG. 8 shows the bottom or caudal plane of an implant from the anterior perspective, having serrations of different heights.

FIGS. 9-10 show side plan views of an implant having serrations of different heights and with different angles of inclination.

FIG. 11 shows a side view of an implant with top serrations in a linear pattern.

FIG. 12 shows a side view of an implant with linear serrations of different heights.

FIG. 13 shows a top view of an implant with linear serrations of different heights.

FIG. 14 shows a bottom view of an implant with linear serrations of different heights.

### DETAILED DESCRIPTION OF THE INVENTION

Exemplary embodiments of the invention will be best understood by reference to the drawings, wherein like parts are designated by like numerals throughout. It will be readily understood that the components of the invention, as generally described and illustrated in the Figures herein, could be arranged and designed in a wide variety of different configurations. Thus, the following more detailed description of the embodiments of the apparatus, system, and method, as represented in the Figures, is not intended to limit the scope of the invention, as claimed, but is merely representative exemplary of exemplary embodiments of the invention.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The term “adjacent” refers to items that are physically near or next to one another and may or may not be in physical contact. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard anatomical reference planes and spinal terminology are used in this specification with their customary meanings.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

Biomechanical models have been constructed to assess slippage due to instability caused by spondylolisthesis. These studies have applied either pure moments or pure shear forces to demonstrate the resulting instability between the segmental units. Other investigators have simulated more realistic physiological loading conditions of the spine by using combined compressive loads and shear forces to evaluate specimen stiffness and range of motion. Compressive loads that have been applied have ranged from 300-1200 N, and shear forces, from −50 N (posterior) to 250 N (anterior). Other studies have evaluated instability due to spondylolisthesis and various methods of surgical fusion in cadaveric spines after creating a simulated injury model. Some of the stabilization (fusion) techniques and implants that have been evaluated include pedicle screws, interbody screw material (titanium v. stainless steel), ALIF cages, and H-plate locking systems. The majority of these studies have used non-destructive testing methods to quantify range of motion in all three planes under three conditions: (1) intact, (2) following simulated spondylolisthesis instability, and (3) after instrumented fixation/fusion. Many of these studies have applied pure moments between 2.5-8 Nm to measure flexion/extension, lateral bending, and axial rotation. Alternatively, some studies have applied a combination of compressive and bending loads off-axis to simulate spondylolisthesis physiological loading. In one study, a 500 N load was applied 2 cm anterior, posterior, and lateral to the center of rotation to simulate this off-axis loading. Preloads between 100-400 N were also applied before testing. Differences in range of motion have been used to draw conclusions about differences in construct stability. Overall, the greatest stability (lowest range of motion) has been achieved in spondylolisthesis models when posterior fixation is used in conjunction with another fixation modality.

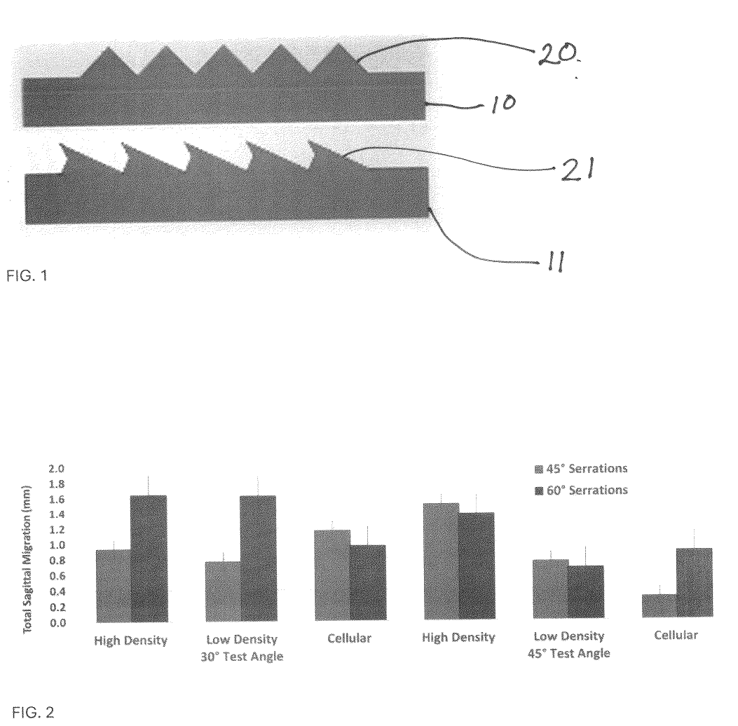

FIG. 1 depicts the side plan view of two different and exemplary implants. 10, 11 with serrations at different angles relative to the surface of the implant. Implant 10 is depicted with serrations 20 rising from the surface at 45 degrees. Implant 11 depicts serrations 21 rising from the surface at 60 degrees. The shape of the serrations, including the base attached to the surface of the implant and the peak or tip, which would contact bone or other substrate may have the profile as shown in FIG. 1, or another shape dependent on the anatomical substrate and desired strength.

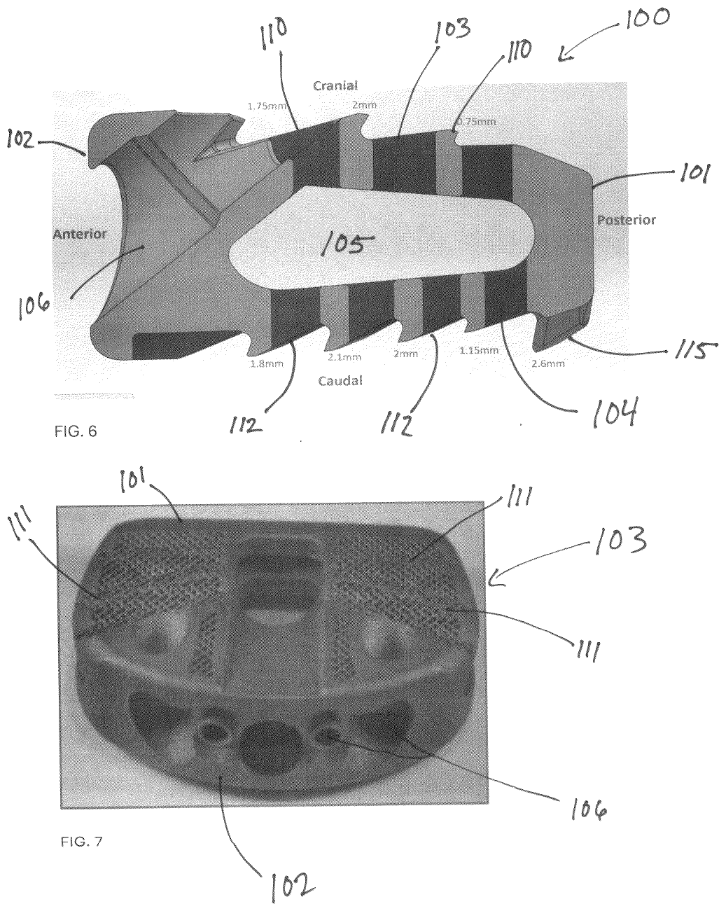

As shown in FIG. 2, two implant surface architectures, based on those depicted in FIG. 1, were evaluated under simulated physiological loading conditions using composite model bone blocks. Each of the two implant serration designs were tested separately against three synthetic bone models of varying density and surface architecture, specifically; High Density (S016): 10 PCF solid rigid polyurethane foam of 0.16 g/cm³, Low Density (S008): 5 PCF solid rigid polyurethane foam of 0.08 g/cm³, and, finally Cellular (C016): 10 PCF cellular rigid polyurethane foam of 0.16 g/cm³. Two implant surface architectures, represented by the exemplary FIG. 1, were fabricated from stainless steel; both had 2 mm height serrations, one with 45° and the other with 60° angles from the transverse plane of the device. Spondylolisthesis loading was simulated using a custom apparatus built into an MTS 858 mini-bionix servo-hydraulic load frame (MTS Systems, Minneapolis, MN), as depicted in FIG. 6.

A cyclic load profile with an amplitude of 100N, increasing peak in increments of 100N after every 10 cycles, was applied at a rate of 0.5 Hz in a step-wise fashion until failure. Shear implant-bone micromotion was measured using a D2/200A linear variable differential transformer (LVDT) while force and actuator displacement were also recorded. Each implant serration design was evaluated three times, using separate bone blocks on each of the three bone analog types, and repeated at both a 30° and 45° sagittal inclination, for a total of 36 experimental specimens. Student's t-tests were used to compare the sagittal migration and maximum force at failure as a function of sagittal inclination and bone analog type. A total of 36 experiments were done.

In terms of total sagittal implant migration, the 45° serrations outperformed the 60°. In trials with a similar max force, the 2 mm, 60° implant had consistently higher sagittal displacement. Trials isolated to 30° inclination with high density foam had largely consistent max forces, but the 2 mm, 60° implant underwent 1.64 mm average migration while the 2 mm, 45° implant underwent a mean of 0.93 mm of migration. At the 30° sagittal inclination, implant serration design played a role in max force sustained at the interface and total migration, while bone analog quality was largely involved in max force sustained at the interface. When sagittal inclination was increased to 45°, implant serration design no longer played a role in sagittal displacement and had a lesser, but still statistically significant, impact on max force at the interface. Multiple trials at the 45° sagittal inclination resulted in near immediate failure at the interface, particularly for the low density foam and the cellular bone analog models.

In summary, the maximum force of failure at the implant-bone interface was highly dependent on surrounding bone analog quality and sagittal inclination. In contrast, overall, the maximum force prior to failure was not influenced significantly by implant design (serration angles).

In terms of limiting total sagittal migration, the 45° serration was more stable than the 60° serrations at 30° of inclination. In trials with similar max force sustained at the interface, the 2 mm, 60° surface model exhibited consistently higher sagittal migrations. For example, trials isolated to 30° inclination with high density foam had largely consistent max forces, but the 2 mm, 60° implant underwent significantly more migration compared to the 2 mm, 45° implant. When the angle of simulated sagittal inclination was increased to 45°, the surface architecture began to have less influence on sagittal migration, while bone quality appeared to have more influence. The max force sustained at the interface was significantly dependent on the surrounding bone analog quality and severity of sagittal inclination, while implant design had a larger influence on maximum force sustained at a lower angle of simulated sagittal inclination. In summary, at the higher angle of inclination, implant surface architecture no longer played a significant role in sagittal migration and played a much smaller role in max force at the interface. Further, multiple trials at 45° of sagittal inclination resulted in near immediate failure in synthetic bone models representing poor bone quality. The results raise concerns regarding the performance of interbody fusion devices when used in poor quality bone and/or high angle of sagittal inclination. Failure in these cases with low force-to-failure could result in damage to the bony endplates even with separate methods of fixations such as screws.

Multiple trials at the 45° sagittal inclination resulted in near immediate failure at the interface, particularly for the low-density foam and the cellular bone analog models. In summary, the maximum force of failure at the implant-bone interface was highly dependent on surrounding bone analog quality and sagittal inclination. In contrast, overall, the maximum force prior to failure was not influenced significantly by implant design (serration angles).

Previous studies have shown bone quality at the endplate to be a prominent factor in interbody fusion device stability. However, few experiments evaluating device stability have considered the influence of bone quality beyond initial cadaveric specimen characterization. Accordingly, previous in vitro models have either used cadavers with comparable BMD or synthetic polyurethane bone models to eliminate bone quality as a variable. It is asserted that bone quality is a variable of interest as stabilizing features in implant and device design, such as angled serrations, may perform differently or have different advantages in different quality of endplates. At lower angles of simulated sagittal inclination, implant serration design and bone analog quality played significant roles in implant stability, with the 45° serrations providing greater stability. However, with the higher angle of spondylolisthesis sagittal inclination, bone analog quality had near total influence over implant stability. The results raise concerns regarding the performance of interbody fusion devices when used in poor quality bone and/or high angle of sagittal inclination.

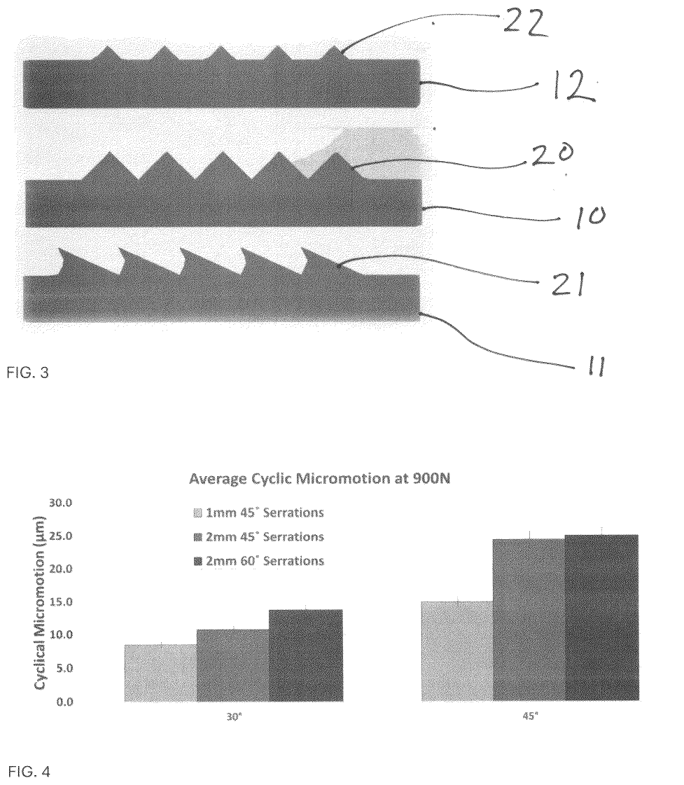

Three implant surface architectures, as depicted in FIG. 3, were evaluated under simulated spondylolisthesis loading conditions using composite model bone blocks (10 PCF solid rigid polyurethane foam, 0.16 g/cm 3, Sawbones, Inc., Vashon Island, WA). The 3 architectures were 1 mm, 45° serration (120, 122), 2 mm, 45° serration (100 102), and 2 mm, 60° serration (110 112), (N=6 each, total N=18). Spondylolisthesis loading was simulated using an MTS 858 mini-bionix servo-hydraulic load frame equipped, as in FIG. 5, with a custom apparatus to simulate 30° and 45° of lumbar spondylolisthesis sagittal inclination (MTS Systems, Eden Prairie, MN). A cyclic load profile with an amplitude of 100N, increasing the maximum force in increments of 100N after every 10 cycles was applied at a rate of 0.5 Hz in a step-wise fashion until failure. Shear micromotion of the implant relative to the bone was measured using an LVDT. Interfacial motions were examined and compared under 800-900N of cyclic loading, representing physiological loading conditions. All three implant designs were evaluated at a 30-degree sagittal inclination (N=3 each, total N=9) and then at a 45-degree sagittal inclination. A univariate ANOVA was performed to examine the overall dataset (N=18) as well as a separate ANOVA for the 30-degree and 45-degree sagittal inclination, respectively (N=9).

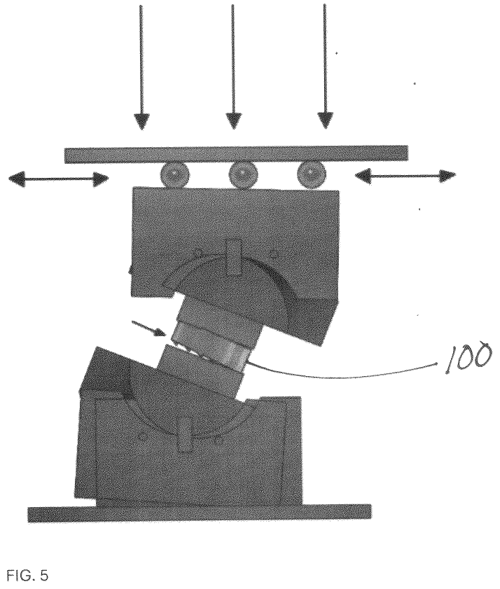

The results for the micromotion test are shown as a bar chart in FIG. 4. Overall, the 1 mm 45° serrations 122 exhibited the least micromotion, regardless of degree of sagittal inclination (p<0.01). This difference was more pronounced at 45 degrees of sagittal inclination, than at the 30-degree inclination. All three device designs were more stable (less micromotion) at a 30-degree sagittal inclination, than at 45 degrees of simulated spondylolisthesis. At a sagittal inclination of 30 degrees, the average cyclic micromotion was 8.4 μm for 1 mm, 45° serrations 122, 10.7 μm for the 2 mm, 45° serrations 102, and 13.7 μm for the 2 mm, 60° serrations 112. At a test angle of 45 degrees, the average cyclic displacement was 14.9 μm for 1 mm, 45° serrations 122, 24.3 μm for the 2 mm, 45° serrations 102, and 24.9 μm for the 2 mm, 60° serrations 112. Further, when comparing the cyclic displacements of both 2 mm surface architectures 122, 102 at the 30-degree sagittal inclination, serration angle could be compared directly, and the 2 mm 45° implants 100 had significantly lower micromotion than the 2 mm 60° implants 110 (10.7 μm compared to 13.7 μm, p=0.02). However, when the angle of sagittal inclination was increased to 45 degrees, the differences in micromotion due to serration angle for the same implant designs were much smaller, and not significant (24.3 μm and 24.9 μm respectively, p=0.70).

Previous ALIF designs have compared widely different methods of fixation including different anchors, single height and angle serrations, stabilizing wings, etc., but none have compared serrations of varying height and angle. At physiological loading levels of 800 N to 900 N when examining the overall dataset, smaller height serrations resulted in less cyclic micromotion. One explanation for this observation is that the larger surface area for the 2-mm height serration device resulted in greater integration with the bone analog, which under cyclic motion, resulted in more damage to the bony interface. At the 30° angle of sagittal inclination, the 2 mm, 45° serrations 102 outperformed the 2 mm, 60° serrations 112, but this did not remain consistent when the sagittal inclination was increased to 45°. This could be due to the tip of the 60° serration being oriented closer to the direction of shear force and causing the 60° design to cut into the bone analog more than the 45° serrations. The potential advantage of the 45° serrations over the 60° serrations appeared to be less relevant at higher angles of sagittal inclination. One limitation of the present study was that no interbody screws were used for fixation, as would be in a clinical setting. Therefore, future studies should include screw fixation to determine the relative contribution of the screws to the overall construct stability.

The results of this study indicate that the 2 mm height implant serrations produce higher levels of interface micromotion than the 1 mm height device. Future studies are needed to determine the optimal design for interbody fusion devices.

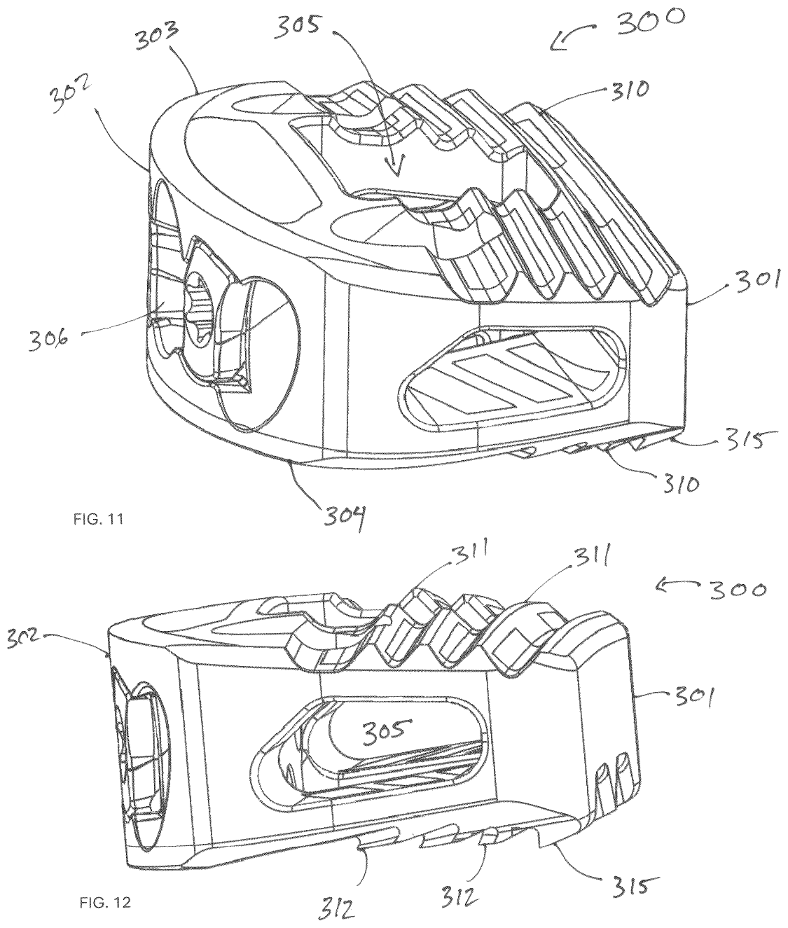

FIGS. 6-15 illustrate by way of example only, intervertebral devices or implants for implantation between two adjacent vertebral bodies. The different embodiments of the implant have dimensions of height, width, and length suitable for placement between vertebral bodies. In the embodiments, the height extends along a cephalad-caudal or superior-inferior direction, the width extends along a right-left direction, and the length extends along an anterior-posterior direction. The embodiments of the implant may be made of any suitable biocompatible material. Various biocompatible materials contemplated include, but are not limited to, poly-ether-ether-ketone (PEEK), other polymers including bioresorbable polymers, ceramics, composites, bone or bone substitute materials, and biocompatible metals including stainless steel, titanium, or tantalum and their alloys. The implants may also include multiple and combinations of the materials. The implants may be manufactured by known methods such as machining, molding, forming, or 3D printing. The implants may be provided in any number of shapes or sizes depending on the specific surgical procedure, need, or patient anatomy. The implants may contain separate radiographic markers of any size of shape suitable to facilitate effective and accurate visualization of implant placement, necessary depending on the base material of the implant.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

Referring to FIGS. 6-8, an embodiment of an implantable device 100 with variable height serrations is illustrated. The device 100 has a general structure including a posterior side 101 opposite an anterior side 102, a top or superior side 103 opposite a bottom or inferior side 104. The posterior 101, anterior 102, top 103, and bottom 104 sides generally contain a central space 105. The central space may be an open void or a low-density and compressible material. The central space may be large enough so that the device may be essentially hollow. Any of the sides of the device 100 may be constructed of or have sections constructed with perforations to allow bone in-growth. The device 100 may have apertures 106 which can be configured for anchor screws or other known means for attachment to surrounding bone.

The device 100 includes ridges or serrations 110 on the top 103 and bottom 104 sides to engage with the surrounding tissue once with device is implanted. The embodiment in FIG. 6 depicts a series of serrations 110 on the top side 103 that are directed posteriorly and a series of serrations on the bottom side 104 that are directed anteriorly. The top serrations 111 are positioned opposite the bottom serrations 112 to improve stability of the device 100 after implantation between adjacent vertebrae. The top serrations 111, discourage posterior migration and the bottom serrations discourage anterior migration. In the embodiment, serrations 110 extend at an approximate 60 degree angle from the transverse plane of the device 100. Depending on the orientation of the top side 103 and bottom side 104 surrounding each serration, the relative angle of the serration 110 may appear to be greater or less than 60 degrees. However, the angle of the serration 100 is determined by the transverse plane of the device 100, which is generally parallel to the base plate of the adjacent vertebrae once implanted. Therefore, the angle of the serrations 110 remain consistent relative to the vertebrae.

The device 100 may include serrations 110 having various heights along the top 103 and bottom 104 sides. In FIG. 6, the device is shown with three top serrations 111 with heights of 1.75 mm, 2 mm, and 0.75 from an anterior to posterior location. The heights are measured relative to the top surface 103 adjacent to the portion of the top serration 111 having the 60 degree angle. The anterior-posterior spacing between the top serrations 111 may deviate from what is depicted in FIG. 6 based on the anatomy of a patient or the requirements for a successful surgery. The number of top serrations 111 may deviate from what is depicted in FIG. 6 based on the anatomy of a patient or the requirements for a successful surgery.

In FIG. 6, the device is shown with five bottom serrations 112 with heights of 1.8 mm, 2.1 mm, 2.0 mm, 1.15 mm, and 2.6 from an anterior to posterior location. The posterior-most serration has the greatest height so that it acts as a posterior anchor, thereby enhancing posterior fixation of the device 100. The heights are measured relative to the bottom surface 104 adjacent to the portion of the bottom serration 112 having the 60 degree angle. The anterior-posterior spacing between the bottom serrations 112 may deviate from what is depicted in FIG. 6 based on the anatomy of a patient or the requirements for a successful surgery. The number of bottom serrations 112 may deviate from what is depicted in FIG. 6 based on the anatomy of a patient or the requirements for a successful surgery. It is within the scope of this invention that the number and location of the top 111 and bottom 112 serrations may be the same or different. The height of the serrations may be the same or different across the span of the top 103 and bottom 104 sides of the device.

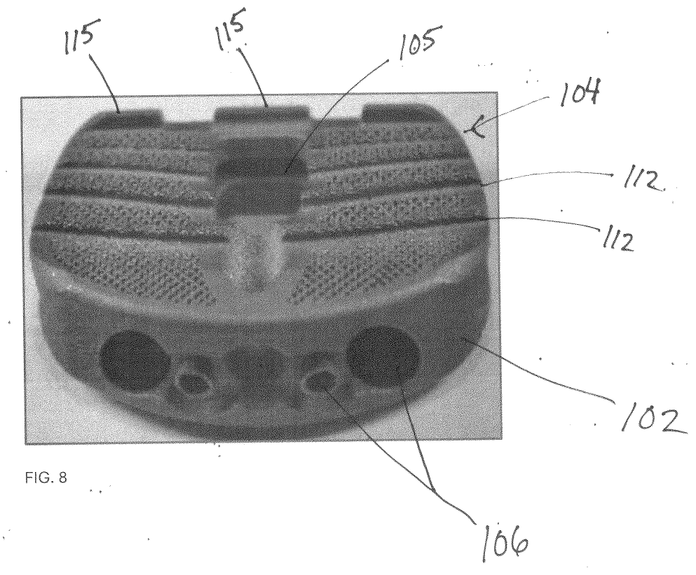

FIG. 7 depicts the top side 103 of device 100. The central space 105 is shown passing through the top side 103 through to the bottom side 104. Apertures 106 are shown as capable of receiving bone anchors or placement devices (not shown). FIG. 8 depicts the bottom side 104 of the device. Device 100 is shown with serrations 110 in a ridge-like formation spanning the width of the device. The serrations 110 may align straight across the top 103 and bottom 104 sides of device, or the serrations 100 may take a curvilinear form or any non-standard orientation, such a repeating zig-zag. The serrations may also be divided into separate teeth across the width of the device. The teeth may be arranged in any pattern necessary for proper fixation and the teeth may have various heights across either or both sides of the device 100.

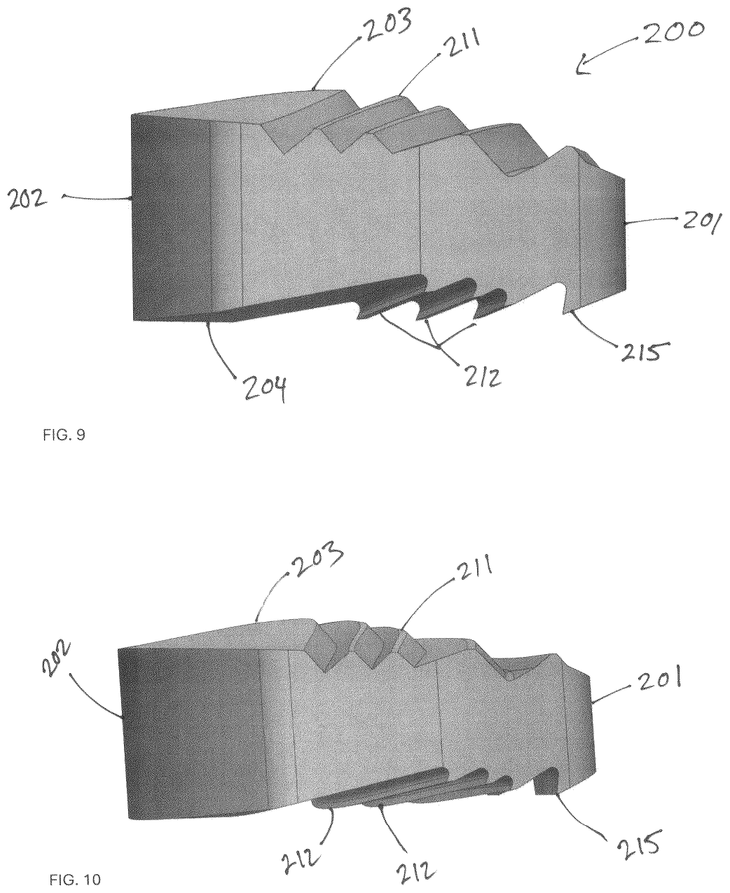

In FIGS. 9-10, an embodiment of the present invention is illustrated. An implantable device 200 with variable height serrations, with varied angles is illustrated. The device 200 has a general structure including a posterior side 201 opposite an anterior side 202, a top or superior side 203 opposite a bottom or inferior side 204. The posterior 201, anterior 202, top 203, and bottom 204 sides may contain a central space (not shown). The central space may be an open void or a low-density and compressible material. The central space may be large enough so that the device may be essentially hollow. Any of the sides of the device 200 may be constructed of, or have sections constructed with perforations, to allow bone in-growth. The device 200 may have apertures configured for anchor screws or other known means for attachment to surrounding bone.

The device 201 includes ridges or serrations 210 on the top 203 and bottom 204 sides to engage with the surrounding tissue once the device 200 is implanted. The embodiment in FIG. 6 depicts a series of serrations 210 on the top side 203 that rise from the top side 203 and a series of serrations on the bottom side 204 that are directed anteriorly. The top serrations 211 are positioned opposite the bottom serrations 212 to improve stability of the device 200 after implantation between adjacent vertebrae. The top serrations 211, discourage general anterior-posterior migration and the bottom serrations 212 discourage primarily anterior migration. In the embodiment, bottom serrations 211 extend at an approximate 60-degree angle from the transverse plane of the device 200. However, the top serrations 211 deviate from the top side 203 at a 45-degree angle. The angle of the serration 200 is determined by the transverse plane of the device 200, which is generally parallel to the base plate of the adjacent vertebrae once implanted. Therefore, the angle of the serrations 210 remain consistent relative to the vertebrae.

The device 200 may include serrations 210 having various heights along the top 203 and bottom 204 sides. The height of the serrations may be within a range appropriate to facilitate successful implantation and prevent migration. FIG. 10 illustrates the top serrations 211 arranged in a curved pattern across the top side 203. The device in FIGS. 9-10 has a different number of top serrations 211 than bottom serrations 212, which may be preferable for implantation in specific patients to prevent migration.

FIGS. 11-14 show yet another embodiment of the present invention. The device has an equal number of top serrations 311 as bottom serrations 312. The device 300 has a general structure including a posterior side 301 opposite an anterior side 302, a top or superior side 303 opposite a bottom or inferior side 304. The posterior 301, anterior 302, top 303, and bottom 304 sides generally contain a central space 305. The central space may be an open void or a low-density and compressible material. The central space may be large enough so that the device may be essentially hollow. Any of the sides of the device 300 may be constructed of or have sections constructed with perforations to allow bone in-growth. The device 300 may have apertures 306 which can be configured for anchor screws or other known means for attachment to surrounding bone.

The device 300 includes ridges or serrations 310 on the top 303 and bottom 304 sides to engage with the surrounding tissue once the device is implanted. The embodiment in FIG. 11 depicts a series of serrations 311 that rise from the top side 303 and a series of serrations 312 on the bottom side 304 that are directed anteriorly. The top serrations 311, discourage anterior-posterior migration and the bottom serrations 312 discourage primarily anterior migration. In the embodiment, bottom serrations 311 extend at an approximate 60-degree angle from the transverse plane of the device 300. However, the top serrations 311 deviate from the top side 303 at a 45-degree angle. The angle of the serration 310 is determined by the transverse plane of the device 300, which is generally parallel to the base plate of the adjacent vertebrae once implanted. Therefore, the angle of the serrations 310 remain consistent relative to the vertebrae.

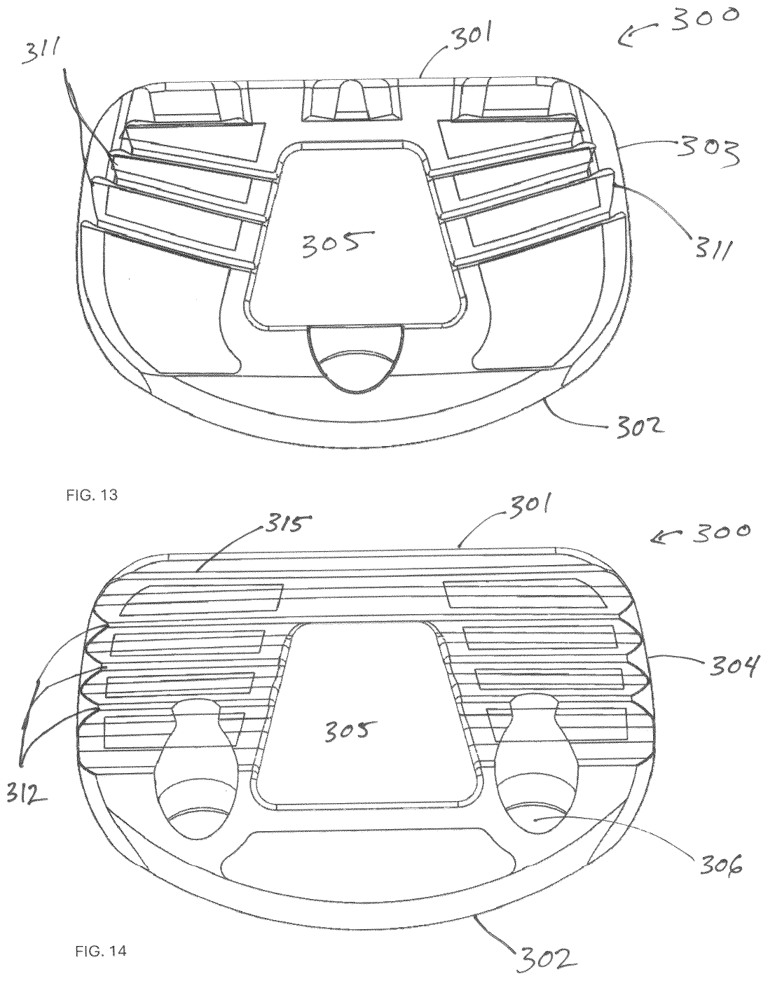

The device 300 may include serrations 310 having various heights along the top 303 and bottom 304 sides. The height of the serrations may be within a range appropriate to facilitate successful implantation and prevent migration. FIG. 13 illustrates the top serrations 311 arranged in an angled chevron pattern from posterior to anterior across the top side 303. FIG. 14 illustrates the bottom serrations 312 arranged linearly and generally perpendicular to the anterior-posterior axis. Any or all of the above referenced serrations may include anti-migration features such as ridges, teeth, lugs, or other purchase-inducing surface treatments.

### EQUIVALENTS AND SCOPE

Those skilled in the art will recognize or be able to ascertain using no more than routine experimentation, many equivalents to the specific embodiments in accordance with the invention described herein. The scope of the present invention is not intended to be limited to the above description, but rather is as set forth in the appended claims.

Any methods disclosed herein comprise one or more steps or actions for performing the described method. The method steps and/or actions may be interchanged with one another and applicable to all embodiments of the intervertebral body implants described herein. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the invention.

While specific embodiments and applications of the present invention have been illustrated and described, it is to be understood that the invention is not limited to the precise configuration and components disclosed herein. Various modifications, changes, and variations which will be apparent to those skilled in the art may be made in the arrangement, operation, and details of the methods and systems of the present invention disclosed herein without departing from the spirit and scope of the invention.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. An intervertebral implant for implantation in an intervertebral space between vertebrae, the implant comprising:
a body extending from an upper surface to a lower surface, the body having an anterior end, a posterior end, and a pair of spaced apart first and second side walls extending between the anterior end and the posterior end, such that an internal chamber is defined within the anterior and posterior ends and the first and second side walls, wherein the body further includes a porous structure configured for bone ingrowth;
the upper surface further comprising a plurality of upper serrations positioned such that the serrations are angled and directed toward the posterior end of the body,
wherein the plurality of upper serrations has different heights above the upper surface, and
the lower surface further comprising a plurality of lower serrations positioned such that the serrations are angled and directed toward the anterior end of the body,
wherein the plurality of lower serrations has different heights above the lower surface.

2. The intervertebral implant of claim 1, where in the plurality of lower serrations gradually increase in height along the anterior to posterior direction.

3. The intervertebral implant of claim 1, where in the plurality of lower serrations gradually increase in height by 50% or greater along the anterior to posterior direction.

4. The intervertebral implant of claim 1, where in the plurality of lower serrations have a variation of height along the anterior to posterior direction by 50% or greater.

5. The intervertebral implant of claim 1, where in the plurality of lower serrations gradually increase in height along the anterior to posterior direction, such that a posterior-most serration has a height that is at least 50% greater than the height of an anterior-most serration.

6. The intervertebral implant of claim 5, wherein the posterior-most serration has a height that is twice than the height of the anterior-most serration.

7. The intervertebral implant of claim 1, where in the plurality of upper serrations gradually increase in height along the posterior to anterior direction.

8. The intervertebral implant of claim 1, where in the plurality of upper serrations gradually increase in height by 50% or greater along the posterior to anterior direction.

9. The intervertebral implant of claim 1, where in the plurality of upper serrations have a variation of height along the posterior to anterior direction by 50% or greater.

10. The intervertebral implant of claim 1, where in the plurality of upper serrations gradually increase in height along the posterior to anterior direction, such that an anterior-most serration has a height that is at least 50% greater than the height of a posterior-most serration.

11. The intervertebral implant of claim 10, where the anterior-most serration has a height that is twice the height of the posterior-most serration.

12. An intervertebral implant for implantation in an intervertebral space between vertebrae, the implant comprising:
a body extending from an upper surface to a lower surface, the body having an anterior end, a posterior end, and a pair of spaced apart first and second side walls extending between the anterior end and the posterior end, such that an internal chamber is defined within the anterior and posterior ends and the first and second side walls, wherein the body further includes a porous structure configured for bone ingrowth;
the upper surface further comprising a plurality of upper serrations,
wherein the upper serrations rise from the upper surface at a 45-degree angle, and
wherein each of the plurality of upper serrations have different heights above the upper surface, and
the lower surface further comprising a plurality of lower serrations,
wherein each of the plurality of lower serrations extends from the lower surface at a 60-degree angle and are directed toward the anterior end of the body, and
wherein the plurality of lower serrations has different heights above the lower surface.

13. The intervertebral implant of claim 12, wherein the plurality of lower serrations has a variation of height along the anterior to posterior direction of 50% or greater.

14. The intervertebral implant of claim 13, wherein the plurality of lower serrations gradually increases in height along the anterior to posterior direction, such that a posterior-most serration has a height that is at least 50% greater than the height of an anterior-most serration.

15. The intervertebral implant of claim 12, wherein the plurality of upper serrations is comprised of teeth.

16. The intervertebral implant of claim 12, wherein the plurality of lower serrations is comprised of teeth.

17. An intervertebral implant for implantation in an intervertebral space between vertebrae, the implant comprising:
a body extending from an upper surface to a lower surface, the body having an anterior end, a posterior end, and a pair of spaced apart first and second side walls extending between the anterior end and the posterior end, such that an internal chamber is defined within the anterior and posterior ends and the first and second side walls, wherein the body further includes a porous structure configured for bone ingrowth;
the upper surface further comprising a plurality of upper serrations,
wherein the upper serrations rise from the upper surface at a 45-degree angle, and
wherein each of the plurality of upper serrations have different heights above the upper surface; and
the lower surface further comprising a plurality of lower serrations,
wherein each of the plurality of lower serrations extends from the lower surface at a 60-degree angle and are directed toward the anterior end of the body, and
wherein the plurality of lower serrations has different heights above the lower surface;
wherein the plurality of lower serrations has a variation of height along the anterior to posterior direction of 50% or greater and the variation of height increases along the anterior to posterior direction, such that a posterior-most serration has a height that is at least 50% greater than the height of an anterior-most serration.

18. The intervertebral implant of claim 17, wherein the plurality of upper serrations is comprised of teeth.

19. The intervertebral implant of claim 17, wherein the plurality of lower serrations is comprised of teeth.

20. intervertebral implant of claim 12, wherein the plurality of upper serrations is arranged in a non-linear pattern.
